(19) United States
(12) Patent Application Publication
(10) Pub. No.: US 2022/0218864 A1
(43) Pub. Date: Jul. 14, 2022

(54) FAR-UVC DEVICE FOR USE IN INACTIVATING AIRBORNE PATHOGENS

(71) Applicants: PAUL DAVID TRIBBLE, GRAPEVINE, TX (US); PATRICK K. SOUTH, NORTH RICHLAND HILLS, TX (US); CLARENCE WAYNE DUNN, GRAPEVINE, TX (US)

(72) Inventors: PAUL DAVID TRIBBLE, GRAPEVINE, TX (US); PATRICK K. SOUTH, NORTH RICHLAND HILLS, TX (US); CLARENCE WAYNE DUNN, GRAPEVINE, TX (US)

(21) Appl. No.: 17/145,045

(22) Filed: Jan. 8, 2021

(51) Int. Cl. A61L 9/20

(52) U.S. Cl. CPC A61L 9/20; A61L 2209/111; A61L 2209/14; A61L 2209/12

(57) ABSTRACT

A far-UVC device for eliminating airborne pathogens includes a plenum, a fan, an LED array, and a filter. Air enters through an inlet and passes through the plenum until it exits out the outlet. The filter is located within the plenum adjacent to either the outlet or the inlet. The LED array emits a UV light on the passing air to inactivate airborne pathogens. The fan regulates the flow rate of the air through the plenum. The fan maintains a positive flow of air through the device and eventually exhausts the disinfected air out the outlet. One or more baffles are located within the plenum to direct the air flow through the device.

### BACKGROUND OF THE INVENTION

### 1. Field of the Invention

The present application relates to an air purification device, and more particularly to a device for inactivating airborne pathogens such as bacteria, and viruses in personal space, point-of-use applications.

### 2. Description of Related Art

Of recent importance in society is the spread of communicable diseases. Efforts are being taken to minimize transmission in different ways. One area of difficulty is how to eliminate or minimize transmission in society where groups of people are in close quarters. Individuals can spread airborne pathogens to other individuals in proximity through aerosol transmission even if they are asymptomatic. These pathogens can also land on surfaces where they can be spread to others who come in contact with the surface.

Current solutions focus on treatment of an entire volume of defined air as a whole and not on individual vicinities around a user in the defined volume. This broad approach to disinfect the entire volume of air is inferior and potentially counterproductive as it encourages the spread of any airborne pathogen amongst those in the defined volume prior to the actual disinfection. Disinfecting the air in this manner relies on air passing through potentially occupied space to reach the disinfecting device, causing the spreading of the airborne pathogens before they can be inactivated. These solutions are also dependent on proper air circulation and are not effective in regions of poor air circulation.

One work-around is to maintain social distancing, currently defined as separation of six feet or greater. However, this is not always possible or practical, especially in mass transit, waiting rooms, gatherings such as entertainment venues, restaurants, and classroom situations. Redesign of mass transit craft and/or vehicles to allow social distancing or to introduce barriers between personal spaces introduces delays and cost overhead and further economic impact due to costs associated with designing, building, remodeling and recertification among others and delays in returning to prior service levels.

Reducing occupancy to meet social distancing needs leads to reduced revenue and higher operating costs per passenger in mass transit situations. This also holds true for entertainment venues where the number of customers per event is reduced due to social distancing requirements. In classroom situations it results in fewer students per classroom and can render schools unable to provide adequate in-person education for all students.

Although strides have been made with respect to air filtration and treatment, shortcomings remain. Therefore, a need exists for a novel device to address the issue of point-of-use disinfection of personal air space of individuals in close proximity to each other without drawing air that has passed through the personal air space of others, potentially picking up harmful airborne pathogens. A further need exists for the application of this solution to numerous different situations where individuals will be in close proximity with each other.

### BRIEF SUMMARY OF THE INVENTION

It is an object of the present application to provide an ultraviolet germicidal irradiation (UVGI) system for inactivating airborne pathogens including but not limited to bacteria and viruses in a person's local environment introduced through various means including exhalation. The device draws in air from the area surrounding or in the immediate vicinity of a person, irradiates the air using far-UVC 222-nm light, and exhausts the disinfected air. Far-UVC commonly refers to the subset of ultraviolet (UV) spectrum C band light frequencies from 207 to 222 nanometers (nm). This device utilizes far-UVC light nominally in the 222-nm region.

An object of the device is to continually disinfect the air within its treatment envelope, thereby reducing the time airborne pathogens can remain suspended in the air, and as a result reducing the concentration of airborne pathogens in that area. This provides an advantage over other solutions where the airborne pathogens can remain suspended in the air until reaching a disinfection device whose intake is located in a different part of the treatment area or where air flow is inadequate due to poor circulation or other reasons.

The system and device is situated in close proximity to a user and ideally in front of the user where breath is exhaled to assist in capturing the user's air. It operates with people in their operating envelope and does not require the evacuation of the cabin or room for treatment. The air passes through an inlet adjacent to the user and is directed by one or more baffles and flows by an LED array. One or more filters may be used. An air fan is used to force flow of the air through the device. An electronic system is used to optionally regulate the operation of the system. One or more sensors may be used to detect air quality and permit self-regulation of the device/system as a whole. For applications requiring status reporting and/or remote control, an electronic system consisting of an electronic controller and one or more sensors can be added. This addition can permit remote control and status reporting of such parameters.

It is an object of the present application to have the device and system be adaptable for use in many areas of public space. These may extend into but are not limited to areas of transportation, industrial, medical, military, educational, entertainment, food service, and personal individual use. The device is inherently scalable since it is implemented on a per-user basis rather than per square foot basis. This allows for it to be equally applicable to both large and small spaces.

The device is configured to be economical and easily installed in existing locations with minimal impact. The device should reduce spread of pathogens to surface areas because they are drawn into the device while airborne and inactivated before they can settle. This device has broad applicability in the inactivation of airborne pathogens.

It is a further object of the present application that this device does not produce ozone and therefore requires no ozone mitigation. Prior devices produce ozone as a result of the use of longer wavelength UV light.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a schematic of a far-UVC device and system according to an embodiment of the present application.

FIG. 2 is a representative functional view of the device and system of FIG. 1.

FIG. 3 is a top view of an LED array in an exemplary embodiment of the device and system of FIG. 2.

FIG. 4 is an enlarged front view of the LED array in FIG. 3.

FIG. 5 is a front view of the embodiment of the device and system of FIGS. 3 and 4.

FIG. 6 is a side view of the device and system of FIG. 5.

FIG. 7 is a view of ballasts used to power the LED array in the device and system of FIG. 5.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, a far-UVC device and system 101 is configured to inactivate airborne pathogens such as bacteria, and viruses in personal space, ideally with point-of-use applications. The air is disinfected in an individual's personal space at or near the exhalation point and does not have to pass through other people's space for treatment. Air that escapes the envelope of personal space without being disinfected (such as from a violent cough or sneeze) can be picked up by an adjacent or nearby device and disinfected in that space. Thereby neighboring devices act as supplemental disinfecting devices for air to eliminate further spread of any airborne pathogen.

Device 101 includes the use of LED arrays that are small, safe, semi-conductor devices that can be mass produced economically. This device is small and compact due to the use of smaller and more compact components. These LED arrays provide advantages over prior devices due to their large and bulky size, in part due to their purpose to sterilize a full room and their use of large, bulky components such as mercury lamps.

The LED arrays are not breakable glass lamps which often containing mercury. This eliminates the concerns over exposure of hazardous components when broken. The arrays utilize 222-nm wavelength far-UVC light which is accepted as safe and not in need of mitigation devices for safe human exposure. This is an advantage over prior devices using longer UV wavelengths that are dangerous to humans due to risks including but not limited to skin cancer, and damage to eyes. Devices using these longer wavelengths must include provisions to mitigate these risks. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

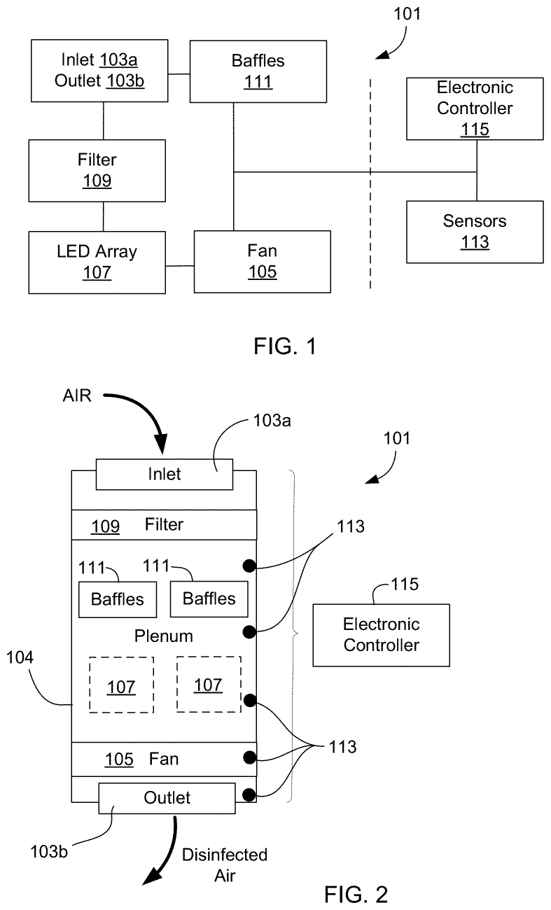

Referring now to FIG. 1 in the drawings, a schematic of a far-UVC device and system 101 is illustrated. Device 101 is configured to capture air for the purpose of inactivating airborne pathogens such as bacteria, and viruses in personal space, point-of-use applications. Device 101 acts as a localized disinfecting system within a larger volume of space. Device 101 includes a plenum 104 (see FIG. 2) that has an inlet 103a and an outlet 103b. Air is drawn through the plenum via a fan 105. The air is passed across an LED array 107 and optionally through a filter 109. Baffles 111 are used to direct the air in the plenum. One or more optional sensors 113 may be used to monitor the air and/or track performance of the overall components in the device 101 and system. An optional electronic controller 115 may be included to regulate performance and communicate captured data to interested parties.

As seen in FIG. 1, controller 115 is operable with any or all of the components of device 101. Device 101, either alone or together with optional controller 115, acts to form the system used to inactivate airborne pathogens. It is understood that the particular quantities of each component are not limited to a singular or plural amount and that the illustrated quantities are not meant to be limiting. Device 101 may include any number of components necessary to fulfill its intended purpose.

Referring now also to FIG. 2 in the drawings, a representative functional view of device 101 is illustrated. As seen, air enters through inlet 103a and passes through plenum 104. The air passes through optional filter 109 and through the UV light emitted via array 107. The baffles 111 are located within plenum 104 and are used to direct the air flow by array 107 through the device 101. The fan 105 maintains a positive flow of air through device 101 and eventually exhausts the disinfected air out outlet 103b.

The specific location of each component within the device may vary with application. Different applications may center around transportation industries, buildings, and industrial applications to name a few. Transportation uses can be and are not limited to aircraft, and buses and other vehicles such as cars and trucks and tractor trailers. Building applications may involve seating areas in airports and terminals. Industrial uses can be and are not limited to personal devices worn for hazardous protection in areas of contamination. Medical uses can be and are not limited to doctor's offices, functions on or near hospital beds, the use by medical personnel in self-contained protection gear, and the possible use by family or guardian members to attend close-by hospitalized patients. Military uses can be and are not limited to the use by military personnel individually or in all types and manner of military vehicles. In educational surroundings the uses can be and are not limited to individual student desks and tables where they work and congregate. It can also be used by teachers at their desks and tables. Any industry may have a use for device 101 and each application may dictate some variations in location for each component.

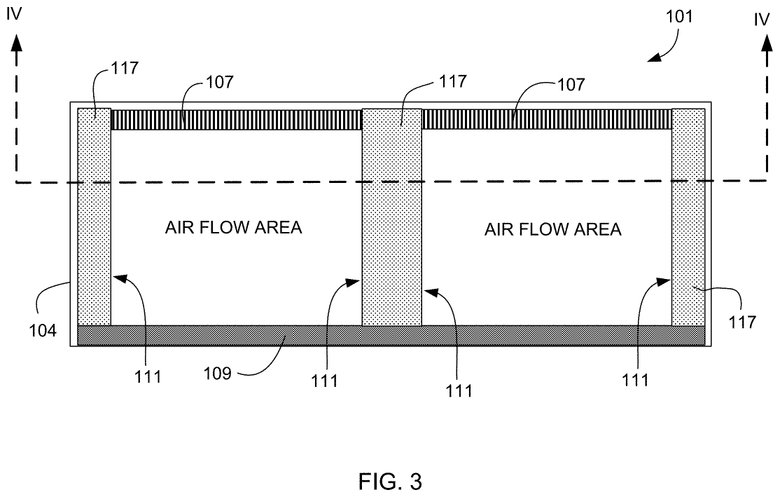

Optional filter 109 is ideally located at one end of plenum 104 while exhaust air fan 105 is located at the other end of the air plenum 104. LED arrays 107 and their mounts 119 should be located within the air plenum between the input air filter 109 and the exhaust air fan 105. In order to direct air across the light emitting portion of LED arrays 107, air baffles 111 should be positioned between the input air filter 109 and the exhaust air fan 105 side of the LED arrays 107. Air baffles 111 create multiple air flow paths through the air plenum 104. The air baffle caps 117 are located as indicated in FIG. 3 to constrain air flow to only those paths that cause air to flow across the LED arrays 107. Air flowing across LED arrays 107 is irradiated by light strong enough to inactivate airborne pathogens.

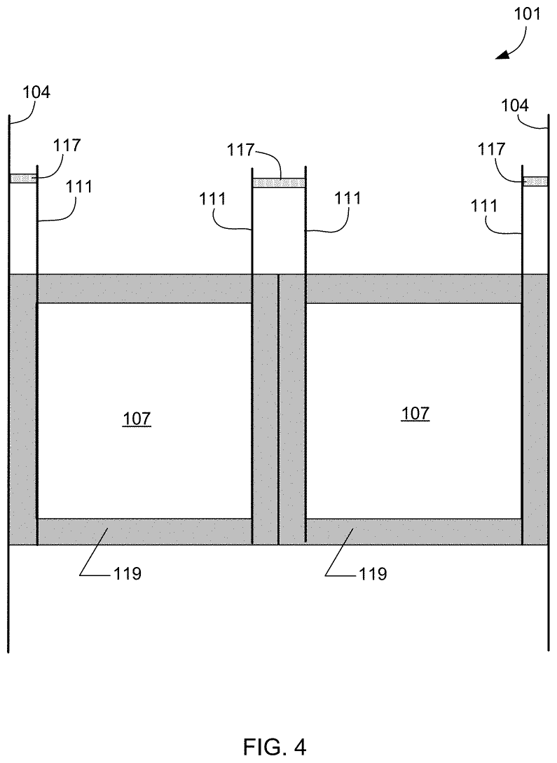

Referring now also to FIGS. 3 and 4 in the drawings, an exemplary view of two LED arrays 107 within plenum 104 is illustrated. FIG. 3 is a top view to show a representative embodiment of device 101. FIG. 4 is a front sectional view showing two arrays 107 from FIG. 3. Optional filter 109 is shown covering at least a portion of the air flow area. This is appropriate if and when the inlet 103a is from a side of the plenum as opposed to the top as shown in FIG. 2. The baffles 111 are coupled to the plenum 104 and optionally to any portion of the interior sides of the plenum 104. Air baffle caps 117 constrain air flow to the air flow areas as shown in FIG. 3. The arrays 107 are situated to one side of an interior surface of the plenum 104.

The air baffle caps 117 are configured to direct air through the defined air flow areas so all air passes through the UV light emitted from the light emitting region of each LED array 107. These air flow areas are irradiated by light from the LED arrays 107 with sufficient energy to inactivate pathogens that are harmful to humans.

Also of note in FIG. 3 is the incorporation of dual air flow areas. Plenum 104 may include one or more air flow areas that may act independent from one another or in combination with respect to each other. Each air flow area is shown to have an array 107. In this depicted embodiment, the air flow areas are side by side. The arrays 107 are also side by side. The orientation of the arrays may differ in that they may be on opposite sides of plenum 104, they may be one above the other, or in any other relative position when compared to other arrays.

Device 101 contains any number of LED arrays 107, as driven by the requirements of each application. As an example, an application requiring a small amount of air flow might contain only one LED array whereas an application requiring a large amount of air flow might contain numerous LED arrays. Likewise, the height, width, thickness, and type of material used to construct the device also varies between applications. Arrays 107 are mounted via mount 119 to an interior wall of the plenum 104. The mount 119 may take any shape sufficient to ensure the stability of array 107 to the plenum surface. The mount 119 is configured so as to avoid obstruction of the UV emitted lights from each array 107 where possible.

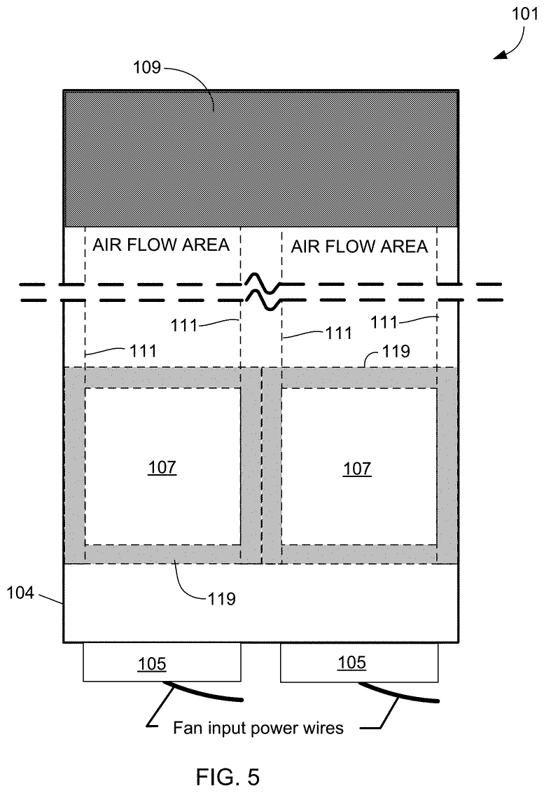

Referring now also to FIG. 5 in the drawings, a front view of device 101 as seen from FIGS. 3 and 4 is illustrated. FIG. 5 shows the optional input air filter 109 at the top and air exhaust fans 105 at the bottom, but this orientation can change in accordance with requirements of a specific application. Also, some applications may require contoured air plenum 104 walls rather than the straight walls shown in these figures. Louvers or additional duct work to direct air flow into or out of the device 101 may be added before the input air filter 109 and/or after the air exhaust fans 105, if required by a specific application.

FIG. 5 is an elevation view of the device that illustrates how air enters through the input air filter 109, flows through the air plenum 104 within the air flow areas defined by the air baffles 111. The air and pathogens then flow across the active light emitting regions of the LED arrays 107 and exits through the air exhaust fans 105. Fan input power wires can be either plugged into or directly connected to any appropriate and convenient power source, such as optional controller 115. The optional input air filter's primary purpose is to reduce the potential for particles such as dust and debris of entering the air plenum 104 and to protect the components of the device. It is understood that filter 109 may constrain some airborne pathogens in some embodiments. It is also understood that filter 109 may not be used in some embodiments.

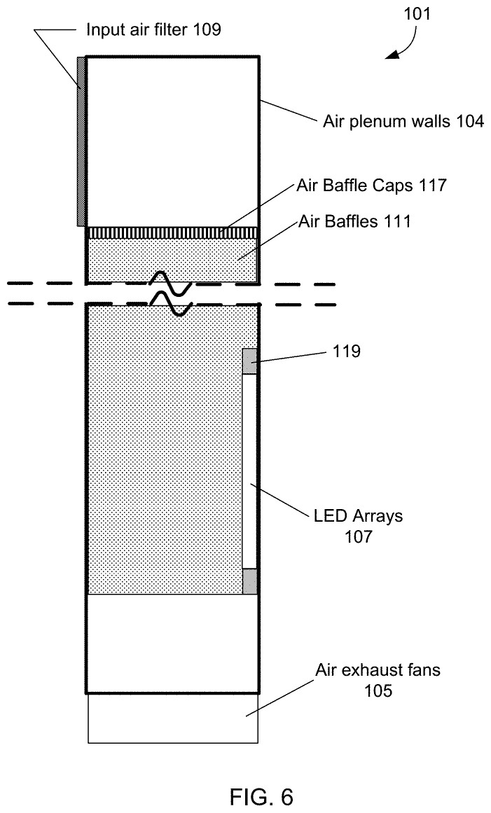

Referring now also to FIG. 6 in the drawings, a side view of the device as seen in FIG. 5 is shown. FIG. 6 is a side view of the device and provides a clear view of the air path from input air filter 109 across the LED arrays 107 and through the air exhaust fans 105. The elevation break shown on this figure illustrates that the elevation as well as other dimension of this device can vary with application requirements.

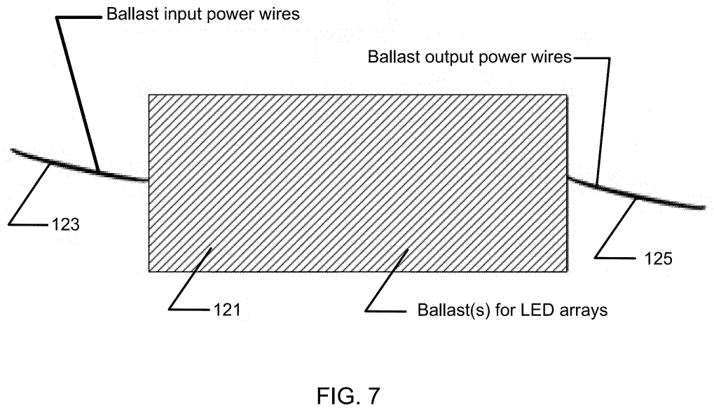

Referring now also to FIG. 7 in the drawings, a front view of ballasts is illustrated. FIG. 7 is a view of the ballast(s) 121 that serve as a power supply for the LED array(s) 107 and may be incorporated into controller 115. Ballasts 121 can be mounted at any convenient location near the air plenum 104. Also shown are the ballast input wires 123 and output power wires 125, respectively. The input power wires 123 can be either plugged into or directly connected to any appropriate and convenient power source, depending on the specific application. Depending on the application, the ballast output power wires 125 can be either plugged into a connector near the LED array(s) 107 or connected directly to the LED arrays 107. More than one ballast 121 could be required depending on the number of LED arrays required by the specific application.

In operation, a user may turn on or off device 101. Fan 105 affects the speed at which air passes through device 101. The speed of air movement correlates to the time the air has to be disinfected through exposure to the UV light of arrays 107 where it is irradiated by far-UVC light of appropriate frequency and sufficient concentration over a sufficient period of time to inactivate airborne pathogens passing through the device. It is understood that the speed at which air moves through device 101 may be predetermined or set by others than the user. Power levels to arrays 107 may influence the acceptable levels of air speed through device 101. It is understood that device 101 can activate or deactivate one or more arrays 107 to compensate for air speed changes and/or selectively adjust the air flow area.

These descriptions and figures show representative views of device 101. The number of components used in these diagrams and descriptions is one example and should not be regarded as limiting. The number of these components used can vary according to the embodiment and application of the device. This device includes applications with any number of LED arrays and various physical configurations as necessary to satisfy details of each specific application.

The figures illustrate some general schematic and representative views for device 101 and its system. Other figures provide an exemplary view of how it may look in application. The dimensions, spacing, and orientations are dependent upon the particular application. For example, the mass transit application of this device may envision each seating position with its own device whether incorporated into the seat or some partition in front of it, or another suitable location. This allows the aggregation of their coverage areas to provide coverage of the cabin/seating area without drawing air potentially containing airborne pathogens across the cabin.

The distribution of these benefits throughout the cabin/seating area provides a cumulative advantage. This is an advantage over room air devices due to the requirement of the air to cycle through the airspace of the room before returning to the treatment device, thereby requiring any air potentially containing airborne pathogens to travel through potentially occupied airspace potentially exposing other individuals and surfaces before it reaches the disinfecting device. This same advantage exists over devices that disinfect the air as it transits through air ducts since the air to be disinfected must transit potentially occupied space to be drawn into the duct.

In aerospace industries, device 101 may be installed into aircraft seat backs and space dividers. This eliminates the need to adjust current air flow circulation systems. This would cut cost of redesigning and recertifying aircraft and retraining crews. In the case where this device is installed into airline seat backs, there would be a revenue savings from not reducing seat count/occupancy due to not occupying the center seat(s) in a three-seat or more configuration in order to maintain social distancing.

Another option is the inclusion of sensors 113. Sensors 113 may be in communication with controller 115 and configured to monitor and assess different functional aspects of the components of device 101 and/or the air that passes therethrough. As seen in FIG. 2, sensors 113 can be located in or in communication with any component, such as array 107, fan 105, and so forth. The cross-sectional area of the air flow area may be monitored for instance. Additionally, plenum 104 may include one or more sensors which may be directed to air quality and air speed. The effectiveness of arrays 107 may be analyzed in real time and changes to fan speed, power output by arrays 107, and the like may be automatically adjusted. The use of sensors permits the feedback to third party users to assess performance.

As alluded to previously, another embodiment of this device envisions individually contained protective gear allowing a wearer to move freely in environments such as classrooms, hospitals, nursing homes, etc.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

1. A far-UVC device for inactivating airborne pathogens, comprising:
a seat;
a plenum defining an air flow area, the plenum coupled to a portion of the seat;
a fan configured to draw air through the plenum, the fan coupled to the plenum within the air flow area; and
an LED array having one or more lights configured to emit a UV light to inactivate airborne pathogens, the LED array coupled to an interior surface of the plenum within the air flow area;
wherein air drawn by the fan is passed by the LED array.

2. The device of claim 1, wherein the LED array includes a mount configured to secure the LED array to an interior surface of the plenum.

3-4. (canceled)

5. The device of claim 1, wherein a filter is located between an inlet of the plenum and the LED array.

6. The device of claim 1, wherein the fan is adjacent to the outlet.

7. The device of claim 1, wherein the fan is located between a filter and an inlet of the plenum.

8. The device of claim 1, wherein the plenum also includes a filter removably coupled within the plenum and configured to cover the air flow area.

9. The device of claim 1, further comprising:
a sensor in communication with at least one of the LED array and the fan, the sensor configured to communicate with an electronic controller to monitor performance of the at least one of the LED array and the fan.

10. The device of claim 1, further comprising:
a sensor in communication with an electronic controller, the sensor located within the air flow area and configured to assess the cleanliness of the air

11. The device of claim 10, wherein assessment of the cleanliness of the air occurs after the LED array.

12. The device of claim 10, wherein assessment of the cleanliness of the air occurs after and before the LED array.

13. The device of claim 10, wherein assessment of the cleanliness of the air occurs prior to the LED array.

14. The device of claim 10, wherein the speed of the fan is adjusted by the electronic controller in relation to air quality as measured after the LED array.

15. The device of claim 1, further comprising:
a baffle coupled to the plenum within the air flow area, the baffle configured to direct the flow of the air past the LED array.

16. The device of claim 1, further comprising:
an electronic controller configured to communicate data regarding the performance of the far-UVC device to a remote party.

17. The device of claim 1, further comprising:
a sensor in communication with an electronic controller, the sensor monitors the performance of the fan so as to regulate air flow velocity through the plenum.

18. A method of inactivating airborne pathogens, comprising:
obtaining the device of claim 1;
locating the device on a portion of the seat;
initiating the fan to draw air through the plenum;
activating the LED array; and
discharging disinfected air out an outlet after being exposed to the UV light from the LED array.

19. The method of claim 18, further comprising:
controlling the speed of air flow through the plenum in response to data received from one or more sensors configured to monitor performance of the device.

20. The method of claim 18, further comprising:
controlling air quality of the air passing through the plenum in response to data received via an electronic controller.

21. The device of claim 1, wherein the plenum includes an inlet, the inlet is oriented on the plenum so as to face rearward of the seat to air behind the seat.

22. A far-UVC system for inactivating airborne pathogens, comprising:
a plurality of seats within an aircraft;
a plenum defining an air flow area, the plenum coupled to a portion of at least one of the plurality of seats;
a fan configured to draw air through the plenum, the fan coupled to the plenum within the air flow area; and
an LED array having one or more lights configured to emit a UV light to inactivate airborne pathogens, the LED array coupled to an interior surface of the plenum within the air flow area;
wherein air drawn by the fan captures air exhaled by a passenger in the aircraft and passes by the exhaled air by the LED array; and
wherein a portion of the plenum faces rearward from the seat.
